(19) United States
(12) Patent Application Publication
(10) Pub. No.: US 2002/0164598 A1
(43) Pub. Date: Nov. 7, 2002

(54) Method for evaluating and applying an individual's genetic characteristics to determine response to cardiovascular medication therapy

(75) Inventors: Joseph B. Muhlestein, Bountiful, UT (US); John F. Carlquist, Park City, UT (US); Benjamin D. Home, Salt Lake City, UT (US); Robert R. Pearson, Farmington, UT (US)

Correspondence Address: Lloyd W. Sadler, Parsons Behle & Latimer, Suite 1800, 201 South Main Street, Salt Lake City, UT, 84111, US

(73) Assignee: IHC Health Services, Inc.

(21) Appl. No.: 09/848,171

(22) Filed: May 3, 2001

(51) Int. Cl. C12Q 1/68; G06F 17/60; G06F 19/00; G01N 33/48; G01N 33/50

(52) U.S. Cl. 435/6; 702/20; 705/3

(57) ABSTRACT

A process for evaluating the genetic sensitivity of individual patients to medications and to appropriately prescribing or not prescribing medication according to established correlations between the genetic sensitivity and the specific medication is described. In the present preferred embodiment/example, this invention provides a process for determining whether a person, having either cardiovascular disease or cardiovascular risk factors, has a genetic characteristic which would indicate whether the ordinarily prescribed statin medication is likely to be effective or even detrimental.

### BACKGROUND OF THE INVENTION

1. Field of the Invention

This invention relates to medical applications of genetic testing. More specifically, this invention relates to methods for identifying appropriate medical treatments using genetic testing and evaluation.

2. Description of Related Art

A number of associations between genetic characteristics and health problems have been reported. Similarly, the applicability of particular pharmacological treatments to specific health disorders has been reported for some time. Generally, however, prior approaches have failed to combine DNA or RNA testing with a prediction of clinical health events in association with particular medications.

The reader is referred to the following U.S. Patent documents, which may or may not necessarily be prior art to the invention of the present invention, for general background material. Each of these U.S. patents is hereby incorporated by reference in its entirety for the material contained therein.

U.S. Pat. No. 4,772,549 describes polymorphisms in genes related to lipid metabolism, specifically apolipoproteins B, DII, E, and apoAIV.

U.S. Pat. No. 4,801,531 describes an early detection method for arteriosclerosis using genetic analysis to detect polymorphisms shown to be correlated with this disease which are proximal to the apolipoprotein Al (apoAl) and aplipoprotein CIII (apoCIII) gene complex.

U.S. Pat. No. 4,879,114 describes 36 lipid extracts and lipid fractions from mammalian omenta containing mostly lipids are used cosmetically for skin conditions.

U.S. Pat. No. 5,268,465 describes a method of purifying calmodulin-dependent nitric oxide syntheses that provides a homogeneous preparation of the enzyme.

U.S. Pat. No. 5,432,058 describes a method for measuring the ability of a human to absorb cholesterol.

U.S. Pat. No. 5,464,742 describes a process to test the association of an allele and a disease, especially a non-Mendelian disease.

U.S. Pat. No. 5,550,021 describes a method for diagnosing and detecting compulsive disorder susceptibility of an individual.

U.S. Pat. Nos. 5,534,615, 5,571,675 and 5,571,893 describe isolated CHF, isolated DNA encoding CHF, and recombinant or synthetic methods of preparing CHF.

U.S. Pat. No. 5,599,673 describes long QT syndrome genes and the identification of the molecular basis thereof.

U.S. Pat. No. 5,624,806 describes isolated CHF, isolated DNA encoding cardiac hypertrophy factor (CHF), and recombinant or synthetic methods of preparing CHF.

U.S. Pat. No. 5,627,073 describes isolated CHF (also referred to cardiac hypertrophy factor or cardiotrophin-1), isolated DNA encoding CHF, hybridomas and cell lines producing antibodies to CHF, and recombinant or synthetic methods of preparing CHF.

U.S. Pat. No. 5,639,614 describes a genetic mutation within the SR calcium release channel that provides a test for susceptibility to idiopathic dilated cardiomyopathy.

U.S. Pat. No. 5,646,130 describes an oligosaccharide containing about 20 monosaccharide units.

U.S. Pat. No. 5,679,545 describes isolated CT-1, isolated DNA encoding CT-1, and recombinant or synthetic methods of preparing CT-1.

U.S. Pat. No. 5,698,411 describes a method for assaying the activity of an enzyme inside a metabolically active whole cell.

U.S. Pat. No. 5,723,585 describes isolated CHF, isolated DNA encoding CHF, recombinant or synthetic methods of preparing CHF, and a method of purifying CHF.

U.S. Pat. No. 5,747,280 describes a human Vascular IBP-Like Growth Factor polypeptide (VIGF) and DNA (RNA) encoding such polypeptide and a procedure for producing such polypeptide by recombinant techniques.

U.S. Pat. No. 5,750,370 describes a human endothelin-bombesin receptor polypeptide and DNA (RNA) encoding such polypeptide and procedure for producing such polypeptide by recombinant techniques.

U.S. Pat. No. 5,750,826 describes a transgenic non-human animal with alterations in a bradykinin B2 receptor gene prepared by introduction of a gene encoding of an altered bradykinin B2 receptor into a host non-human animal.

U.S. Pat. No. 5,760,206 describes the preparation and use of nucleic acid fragments encoding soybean seed sterol-ACP desaturase enzyme or its precursor to modify plant oil composition.

U.S. Pat. No. 5,763,496 describes a method for the prevention and treatment of arteriosclerosis and its related diseases in mammals.

U.S. Pat. No. 5,767,155 describes a method of suppressing ventricular muscle cell hypertrophy induced by an alpha 1-adrenergic agonist or endothelia, by providing an effective amount of a retinoic acid compound.

U.S. Pat. No. 5,780,263 describes a human Small CCN-Like Growth Factor polypeptide (SCGF) and DNA (RNA) encoding such polypeptide and a procedure for producing such polypeptide by recombinant techniques.

U.S. Pat. No. 5,81 7,477 describes a human adrenergic receptor polypeptide and DNA (RNA) encoding such polypeptide and a procedure for producing such polypeptide by recombinant techniques.

U.S. Pat. No. 5,830,430 describes cationic lipid compounds, which comprise at least two cationic groups.

U.S. Pat. No. 5,902,831 describes a method for the prevention and treatment of atherosclerosis and its related diseases in mammals, in which an NADPH oxidase inhibitor is administered.

U.S. Pat. No. 5,916,758 describes isolated nucleic acid encoding human smooth muscle cell-derived migration factor, protein obtainable from the nucleic acid, recombinant host cells transformed with the nucleic acid and use of the protein and nucleic acid sequence.

U.S. Pat. No. 5,916,766 describes human Marco scavenger receptor polypeptides (HMarcoSR) and DNA (RNA) encoding of such HMarcoSR and a procedure for producing such polypeptides by recombinant techniques.

U.S. Pat. No. 5,932,540 describes human VEGF2 polypeptides, biologically active, diagnostically or therapeutically useful fragments, analogs, or derivatives thereof, and DNA (RNA) encoding of such VEGF2 polypeptides.

U.S. Pat. No. 5,942,405 describes human C3a receptor polypeptides and DNA (RNA) encoding such C3a receptor and a procedure for producing such polypeptides by recombinant techniques.

U.S. Pat. No. 5,955,266 describes a method for diagnosing a subject having or at risk of having a thrombotic disease syndrome by analysis of a platelet polymorphism.

U.S. Pat. No. 5,955,443 describes compositions and methods for the treatment and diagnosis of diseases or disorders amenable to treatment through modulation of expression of nucleic acid encoding a platelet endothelial cell adhesion molecule-1 (PECAM-1; also known as CD31 antigen or endoCAM) protein.

U.S. Pat. No. 5,968,981 describes a method of suppressing ventricular muscle cell hypertrophy induced by an alpha 1-adrenergic agonist or endothelin, by providing an effective amount of a retinoic acid compound.

U.S. Pat. No. 5,994,302 describes a human Vascular IBP-like growth factor polypeptide (VIGF) and DNA (RNA) encoding such polypeptide and a procedure for producing such polypeptide by recombinant techniques.

U.S. Pat. No. 5,994,506 describes a human adrenergic receptor polypeptide and DNA (RNA) encoding such polypeptide and a procedure for producing such polypeptide by recombinant techniques.

U.S. Pat. No. 6,013,630 describes methods of using mutant ANF proteins, fragments, analogs, derivatives and homologs of mutant ANF proteins, the nucleic acids encoding these mutant ANF proteins, as well as modulators of ANF for treating or preventing ischemic diseases, in particular ischemic stroke.

U.S. Pat. No. 6,040,157 describes human VEGF2 polypeptides that are biologically active, diagnostically or therapeutically sef1 fragments, analogs, or derivatives thereof, and DNA (RNA) enco such VEGF2 polypeptides.

U.S. Pat. No. 6,056,938 describes cationic lipid compounds, which comprise at least two cationic groups and the use thereof.

U.S. Pat. No. 6,057,332 describes compounds of 1,4-dihydropyrimidine-5-carboxylate compounds and their preparation and uses.

U.S. Pat. No. 6,060,311 describes a method of suppressing ventricular muscle cell hypertrophy induced by an alpha 1-adrenergic agonist or endothelin, by providing an effective amount of a retinoic acid compound.

U.S. Pat. No. 6,071,742 describes the use of artificially attenuated Coxsackievirus B3 cardiotropic virus vectors as efficient gene transfer vectors to deliver immunomodulatory proteins and/or antigenic epitopes.

U.S. Pat. No. 6,111,094 describes compositions and methods for detecting and modulating levels of intercellular adhesion molecule-1 (ICAM-1) proteins, including human ICAM-1.

U.S. Pat. No. 6,117,650 describes an assay to test for hypertropic activity in myocytes.

U.S. Pat. No. 6,132,724 describes an enhancement of attentional processing that is attained by administration of an endorphinase inhibitor or enkephalinase inhibitor and optionally, a dopamine precursor, or a serotonin precursor, a GABA precursor, or an endorphin or enkephalinase releaser, or certain herbal compounds including Rhodiola rosea extract (Pharmaline) and/or Huperzine.

U.S. Pat. No. 6,140,047 describes a method for the early prediction of a propensity to develop chronic obstructive airway disorders such as asthma.

U.S. Pat. No. 6,143,519 describes a human endothelin-bombesin receptor polypeptide and DNA (RNA) encoding such polypeptide and a procedure for producing such polypeptide by recombinant techniques.

U.S. Pat. No. 6,197,931 describes Human Marco scavenger receptor polypeptides (HmarcoSR) and DNA (RNA) encoding such HmarcoSR and a procedure for producing such polypeptides by recombinant techniques.

Also recommended are the following articles, each of which is hereby incorporated by reference in its entirety for the material contained therein. Effect of the Stromelysin-1 Promoter on Efficacy of Pravastatin in Coronary Atherosclerosis and Restenosis, by de Mast et al., published in The American Journal of Cardiology, Volume 83(6), on Mar. 15, 1999, for background material. The Role of a Common Variant of the Cholesteryl Ester Transfer Protein Gene in the Progression of Coronary Atherosclerosis, by Kuivenhoven et al., published in The New England Journal of Medicine, Volume 338(2), on Jan. 8, 1998, which is related to the preferred embodiment/example of this patent disclosure, although it actually ‘teaches away’ from the present invention. Association of Cholesteryl Ester Transfer Protein-Tag 1B Polymorphism with Variations in Lipoprotein Subclasses and Coronary Heart Disease Risk: The Framingham Study, by Ordovas et al., published in Arteriosclerosis, Thrombosis and Vascular Biology, Volume 20(5), in May 2000, for genetic correlation background.

### SUMMARY OF INVENTION

It is desirable to provide a method for predicting the performance and applicability of drug and other medication treatment for patients with specific medical conditions. It is particularly desirable that such a method be based on the application of a genetic test, which provides for the detection of one or more genotypes, the presence of which can be used as a predictor of therapeutic response. In its present preferred embodiment, this invention makes use of the CETP Taq 1B Genotype as a predictor of therapeutic response to statin therapy in patients with severe coronary artery disease.

Accordingly, it is an object of this invention to provide a method for evaluating the genetic sensitivity of individuals to medications.

Another object of this invention is to provide a method for evaluating genetic risk factors and applying such results to the diagnosis and treatment of patients.

A further object of this invention is to provide a method for predicting the performance of medical treatments using genetic sensitivity testing.

A still further object of this invention is to provide a method for determining the genetic sensitivity of individuals to particular medications where the result of a genetic test is compared to established associations.

It is another object of this invention to provide a method for determining the effectiveness of medications where the appropriateness and dosage levels of the medication are determined.

It is a further object of this invention to provide a method for using genetic testing to determine the effectiveness of particular medication treatments that is appropriate for use in a hospital, medical clinic, health maintenance organization, medical office, and similar locations.

It is a still further object of this invention to provide a method for applying the results of genetic testing in the evaluation of medical treatments that combines DNA and RNA testing together with the prediction of clinical events, such as death or myocardial infarction among CV medication users for patents with the CETP Taq 1B polymorphism or ACE gene polymorphism.

A further object of this invention is to provide a method for enabling medical clinicians to determine, based on genetic sensitivities, whether a person will benefit from a particular medication, how much such medication should be prescribed, and does so based on whether specific genetic markers associated with established criteria.

Additional objects, advantages and other novel features of this invention will be set forth in part in the description that follows and in part will become apparent to those skilled in the art upon examination of the following or may be learned with the practice of the invention. The objects and advantages of this invention may be realized and attained by means of the instrumentalities and combinations particularly pointed out in the appended claims. Still other objects of the present invention will become readily apparent to those skilled in the art from the following description wherein there is shown and described the preferred embodiment of this invention, simply by way of illustration of one of the modes best suited to carry out this invention. As it will be realized, this invention is capable of other different embodiments, and its several details and specific steps are capable of modification in various aspects without departing from the concept of this invention. Accordingly, the drawings and descriptions should be regarded as illustrative in nature and not as restrictive.

### BRIEF DESCRIPTION OF THE DRAWINGS

The accompanying drawings incorporated in and forming a part of the specification, illustrate a preferred embodiment of the present invention. Some, although not all, alternative embodiments are described in the following description. In the drawings: FIG. 1 is a process flow diagram of the preferred steps of this invention.

FIG. 2 is a table showing the baseline characteristics of patients categorized by treatment status and by Taq 1B genotype.

FIG. 3 is a chart showing the frequency of events (all-cause mortality and non-fatal MI) for statin-treated and untreated patients further separated into the three possible CETP Taq 1B genotypes.

FIG. 4 is a chart showing the frequency of cardiovascular events (cardiovascular-related death and non-fatal MI) for statin-treated and untreated patients further separated into three possible CETP Taq 1B genotypes.

FIG. 5 is a chart showing subgroup analysis of the frequency of cardiovascular death (A) and non-fatal MI (B) for statin-treated and untreated patients further separated into the three possible CETP Taq 1B genotypes.

FIG. 6 is a table showing patients with nonfatal cardiovascular events during follow-up.

Reference will now be made in detail to the present preferred embodiment of the invention, an example of which is illustrated in the accompanying drawings.

### DETAILED DESCRIPTION

This invention is a method or process for evaluating the genetic sensitivity and benefits of individuals to medications. Although applicable to a wide variety of genetic tests, sensitivities, medical conditions and pharmacological treatments, the present preferred embodiment of this invention, which is presented here as an example of the utility of this invention, is the application of a genetic test which identifies the presence of the CETP Taq 1B genotype and applies this information to the prescription of medication to patients having at least one cardiovascular (CV) risk factor or CV disease. Genetic sensitivity is determined by evaluating the DNA or DNA expression, including RNA, and comparing the result to known associations with the degree of effectiveness or ineffectiveness of the medication to determine whether the medication should be prescribed and, if so, determining the appropriate dosage level of the prescribed medication. In particular, this invention combines DNA or RNA testing together with the prediction of clinical events such as, in the preferred embodiment example, death or myocardial infarction among CV medication users having the CETP Taq 1B polymorphism or ACE gene polymorphism for which CV medication is typically prescribed. Recent research has enabled the inventors to determine that CV patients with these particular genetic traits are not benefited, and may be detrimentally affected, by standard pharmacological treatments. This invention enables clinicians to determine, based on genetic testing, both whether a patient will benefit from a CV medication and, if so, whether and how much medication should be given, and does so for genetic markers associated with the specific laboratory measurements, which CV medications are targeted. By applying this invention, patient care is improved while prescription costs are reduced.

At present, the most common treatment for hypercholesterolemia is the use of 3-hydroxy-3-methylglutaryl-coenzyme A (HMG-CoA) inhibitors (statins). The primary clinical benefits of statin use are reducted coronary events and reduction in overall mortality, presumably related to a reduction of LDL. The cholesterol ester transfer protein (CETP) mediates the transfer of cholesterol esters between the lipid fractions and, thus is a principal regulator of the proportion of lipid fractions in the plasma. Evidence suggests that interactions may occur between statins and CETP activity; and one particular polymorphism in the CETP gene (the Taq 1B polymorphism) may affect the ability of statins to induce lesion regression.

Although the interaction between the Taq 1B genotype has been studied previously, no prior application of the results of a genetic test for this genotype to prescription of statins to CV patients has been proposed.

The inventors have conducted a research study wherein blood was obtained from 2415 consenting patients with severe coronary artery disease, over 70% of which has stenosis, who are undergoing arteriography. Patients were grouped according to statin prescription at hospital discharge and followed for a mean time of 2.4 years; the outcomes of all-cause mortality, cardiovascular (CV) death and non-fatal myocardial infarction (NF-MI). CETP genotyping used PCR amplification of a region in exon 1 and Taq 1 restriction endonuclease digestion. CETP genotype frequencies were B1 B1 32.7%; B1B2 50.3%; and B2B2 17.1% (rounded to the nearest 0.1%). For all combined endpoints, no effective difference in event rate was found between subjects with the B1B1 genotype untreated with statins and treated with statins (12.7% verses 12.2% respectively. Conversely, statin reduced the event rate from 16.3% to 7.7% among B1B2 subjects and from 15.8% to 7.3% for B2B2 individuals. For cardiovascular endpoints (CV death and NF-MI) event rates among B1B1 subjects were 8.2% verses 10.5%, untreated and treated respectively. Indicating that statin treatment for B1B1 subjects may actually increase undesirable events. While CV death and NF-MI event rates among patients positive for the B2 allele were 10.5% untreated with statins, and 6.2% treated with statins. Subgroup analysis revealed that statins did not significantly reduce the rate of NF-MI, although there was a trend toward event reduction among B2 positive patients. Conversely, statins significantly reduced the rate of CV death among B2 positive patients. This study led to the following conclusions. The CETP Taq 1B polymorphism is a high significant predictor of therapeutic outcome of statin therapy in patients with severe CAD. Patients homozygous for the B1 allele do not appear to benefit from statin treatment with regards to all-cause and cardiovascular mortality; conversely, therapeutic benefits of statin treatment is restricted to patients positive for the B2 allele. The application of these findings to the treatment of CV patients, as described in this invention, can have a highly significant impact on health management and the cost of care for patients with severe Cardiac Arterial Disease.

Hypercholesterolemia is a recognized risk factor for atherosclerosis. Presently, the most common medical treatment for elevated plasma cholesterol is the use of 3-hydroxy-3-methylglutaryl-coenzyme A (HMG-CoA) inhibitors (statins). Statins competitively inhibit the conversion of HMG-CoA to melvalonate, the rate-limiting step in cholesterol synthesis. As such, these medications have been found to be effective in lowering plasma LDL concentrations. The principal physiologic benefits derived from the lowering of LDL concentrations include improved endothelial function, which is impaired by the negative effects of elevated cholesterol on NO production, and reduction in the burden of atherosclerotisis. The primary clinical benefits of statin use are reduced coronary events and a reduction in overall mortality. A recent meta-analysis of clinical trials reports an overall 20%-30% reduction in death and cardiovascular events among patients receiving statins. Additionally, the need for therapeutic interventions has been shown to be decreased by statin treatment.

The cholesterol ester transfer protein (CETP) is a key element in the regulation of lipid composition of the plasma. This regulatory function is mediated through the transfer of triglycerides and cholesterol esters between the various lipoproteins. It has been proposed that the CETP mediated transfer of cholesterol ester from the HDL fraction to the LDL and very LDL (VLDL) modifies the proportion of the various lipid fractions in the plasma. Indeed, CETP activity is inversely related to the quality of HDL and a transgenic animal model overexpressing a human CETP gene showed a marked reduction in HDL and developed more severe atherosclerosis than controls. Thus, elevated CETP activity may increase the risk for disease.

Given that statins and CETP both affect plasma lipid concentration and composition, it is not unexpected that there may exist some degree of interaction between them. Indeed, several studies have examined and identified modifying effects of statin therapy on CETP activity. A reduction of CETP activity following the initiation of statin therapy was observed in one randomized trial. This observation was confirmed by researchers who observed a significant reduction in the CETP mediated transfer of cholesterol ester from HDL to apo B-containing lipoproteins among patients on statin therapy. Thus, an expected interaction between statin treatment and CETP activity has been observed.

The CETP gene exists in several polymorphic forms. These polymorphisms are genetic variants that are present in one or more percent of the population. Some of these polymorphisms are not associated with any observable effects on the regulation of gene expression or on the activity of the coded protein, whereas others may exist in regions that regulate gene expression and have functional consequences. One particular CETP polymorphism is the Taq 1B polymorphism in intron 1 of the gene. The B1 allele at this locus is associated with increased mass of CETP. The Taq 1B genotype has been found to be a predictor of lesion progression in response to pravastatin, as determined angiographically. One study has shown that the B1 allele was associated with increased lesion progression. The lesion progression was slowed by pravastatin among patients homozygous for the B1 allele but not for individuals homozygous for the B2 allele. Taken together, these observations point to a stratified risk based on the intron 1 Taq 1B polymorphis and, further, suggest that this locus may predict response to treatment with statins.

In the inventors study and in the application of this invention, DNA is extracted from the EDTA peripheral blood specimens by a standard phenol-chloroform methods. The CETP allelic genotypes arise from the presence or absence of the Taq 1 restriction enzyme site within exon 1 of the CETP gene. Identification of these alleles used polymerase chain reaction (PCR) amplification of a 1412 base pair segment of exon 1 followed by Taq 1 digestion. The sequences for the PCR primers used for the amplification are: forward, 5′ ACA TAT TM GCA ATT ATC CAG AT 3′; reverse, 5′ CAC TTG TGC AAC CCA TAC TTG ACT 3′. Reactions are performed in 50 μL volumes contained 0.1 μm each primer, 100 μm each dNTP, 0.1 0.5 μg genomic DNA, 0.1 U Taq polymerase and 0.1 mg/ml bovine serum. The amplification protocol consisted of an initial denaturation step (94° C. for 5 minutes) followed by 30 cycles, each cycle consisting of a denaturation segments (94° C., 1 minute), an annealing segment (50° C., 1 minute) and 1 minute to allow for extension (72 degrees C.); a final extension at 72 degrees C. for 10 minutes following the last cycle. Amplified products are digested in a digestion mixture of 2 μl reaction buffer (manufacturer), 0.25 μL, 100× BSA, 0.35 μL Taq 1 (20 U/ml, manufacturer), 10 5 μl amplified product to a final volume of 125 μL with sterile distilled water. Digested products were visualized by electrophoresis (2% agarose gel) and staining with ethidium bromide. The presence of two bands (670 bp and 750 bp) indicates a genotype homozygous for the presence of the restriction site (B1B1 genotype). A single band (1412 bp) identifies a genotype in which both CETP alleles lack the Taq 1 restriction site (B2B2 ). A heterozygote (B1B2) is identified by the presence of all three possible bands.

Statin prescriptions at the time of discharge from baseline hospitalization was determined for each subject from an electronic clinical database. A query of this database was made to ask for the prescription of any of the following agents: simvastatin, pravastatin, atorvastatin, lovastatin, fluvastatin, or cerivastatin. Statin use prior to hospitalization was not known in the study, and long-term compliance with therapy was not determined.

The study also considered subject demographics, traditional medical risk factors and clinical variables to determine confounding. These variables included age, gender, lipid levels, diabetes mellitus, hypertension, smoking, family history of CAD, presenting diagnosis, type of clinical treatment of intervention, number of diseased coronary vessels, renal failure, and C-reactive protein (CRP) level. Each of these factors were previously defined in Horne, B. D., Muhlestein, J. B., Carlquist, J. F., Bair, T. L. Madsen, T. E., Hart, N. I., Anderson, J. L., Statin Therapy, Lipid Levels, C-Reactive Protein, and the Survival of Patients with Angiographically Severe Coronary Artery Disease, J.Am. Coll. Cardiol 2000; 36(6): 1774-1780.

The study used the chi-square test or Student's t-test to examine whether a univariate association with mortality or MI exists for each categorical or continuous variable, respectively, including the CETP genotype and the statin prescription status. The association of CETP genotype to mortality or MI was examined for individual differences between the genotypes and any overall trend. The CETP genotype and statin status were evaluated in interaction by the chi-square test.

Evaluation of confounding of CETP genotype or statin prescription status was performed to assess whether any of the other study variables accounted for any observed relationship between the CETP and statin variables and the death/MI outcome. The chi-square test was used for categorical variables and the Student's t-test for continuous variables. Factors with univariate association to the outcome and with univariate association to the CETP genotype or the statin status were considered confounders and evaluated in bivariate logistic regression (using SPSS version 10.0) to determine the magnitude of confounding. Two-tailed p-values are reported with 0.05 designated as the level for nominal significance.

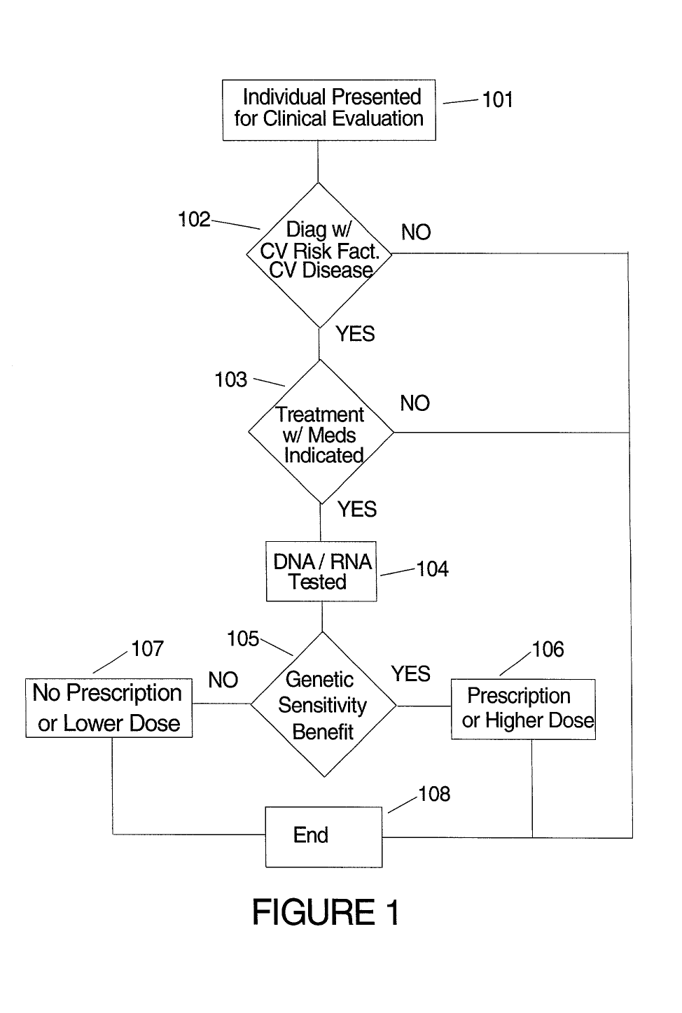

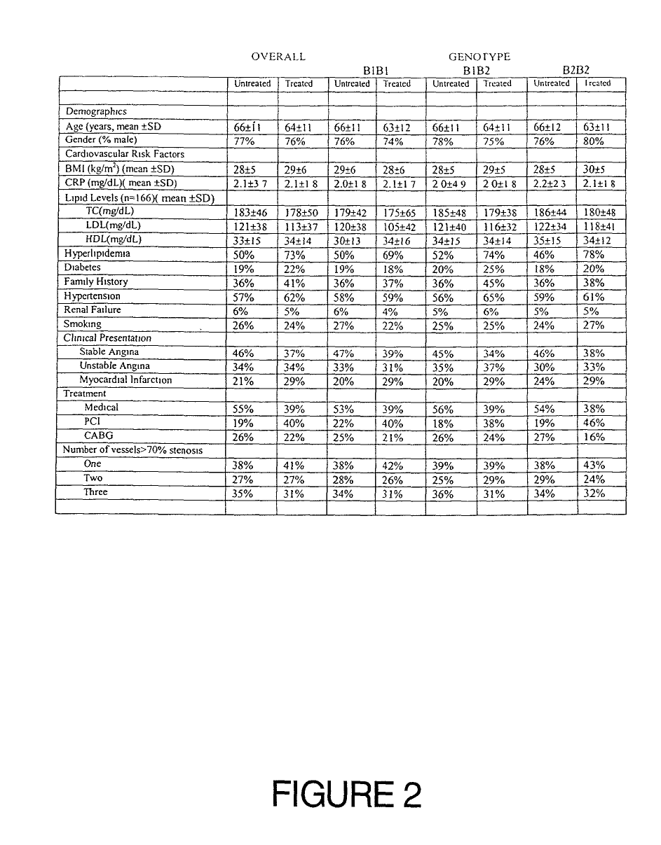

The demographic and key clinical characteristics of the patient sample used in the study are given in the table of FIG. 2. The average age was 64+/−11 years, 76% were male. The distribution of CETP genotypes among the patient population was B1B1, 32.7%; B1B2, 50.3%; and B2B2 17.1%. All allelic frequencies conformed to the Hardy-Weinberg equilibrium. Of the entire patient sample, 675 (28%) were prescribed statin at hospital discharge and 1740 (72%) were not. By specific agent, 70% of prescriptions were for simvastatin, 15% for atorvastatin and the remainder distributed among the remaining agents.

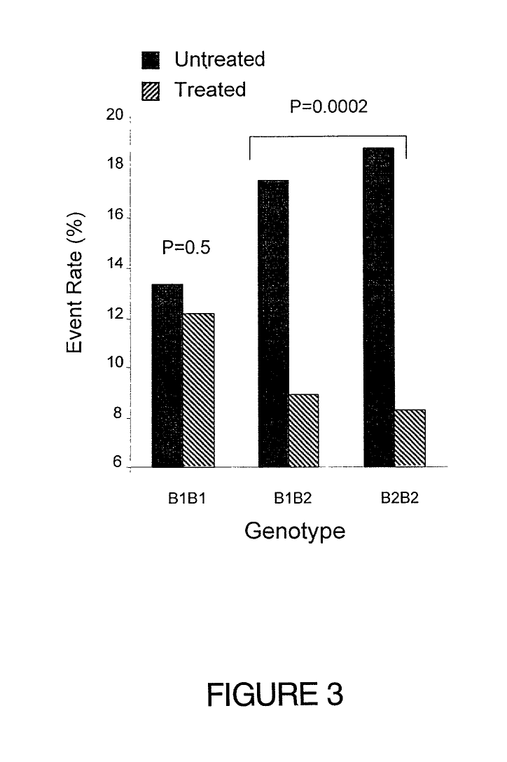

The following study results were observed. Overall, the event rate among treated patients was significantly lower than patients not receiving statins (15.1% versus 9.2%, untreated and treated, respectively; p=0.0001). Patients in each group (treated and untreated) were reanalyzed by CETP genotype for the frequency of events. These results are given in FIG. 3. As presented, no practical difference in endpoint frequency was observed between untreated and treated patients with the B1B1 genotype (12.7% versus 12.2%, respectively, p=0.08). Conversely, for the B1B2 genotype, statin therapy significantly reduced the number of individuals experiencing an event compared to untreated patients (16.3% verses 7.7%, respectively, p=0.0001). Similarly, statin treatment benefited patients with the B2B2 genotype (event positivity: 15.8% verses 7.3% for untreated and treated, respectively; p=0.03). These findings strongly suggest that the possession of a CETP Taq 1B2 allele predicts response to statin therapy with respect to these endpoints. Between the two B2 positive genotypes (B1B2 and B2B2 ) there was found no difference in the rate of death or non-fatal MI suggesting the lack of a gene dosage effect. Patients lacking a B2 allele did not experience therapeutic benefit with respect to the combined endpoints. It is of interest that, for untreated patients, there were actually fewer events among the B1B1 genotype than for patients positive for the B2 allele (12.7% versus 16.2%, respectively), but this only achieved borderline significance (p=0.057). In all, these results point to a mild protective effect for the B1 homozygotes, but the benefit from statin therapy is limited to patients with the B2 allele. These effects were not additive because no difference in event rate was seen between B1B2 and B2B2 individuals. In a similar manner, B2 homozygotes had not therapeutic advantage over B1B2 heterozygotes suggesting that the effect was inherited as a dominant trait.

Furthermore, the study identified the influence of CETP genotype on the benefit of statin therapy with respect to cardiovascular endpoints. A total of 157 deaths occurred during the period of the study. For 123 of those (78.3%) the cause of death was known. Of these 123, 94 (76.4%) were attributed to cardiovascular causes (CV death). To further explore the dependence of therapeutic benefit on the presence of the B2 allele, the inventors specifically examined cardiovascular endpoints (non-fatal MI and CV death) and CETP genotype compared between treated and untreated patients. For cardiovascular events, there was an increase in the event rate for treated B1B1 as compared to untreated patients (10.5% verses 8.2%, respectively) but this result may not have been particularly significant (p=0.29). Conversely, treatment with statin reduced the event rate for B1B2 subjects (6.1% verses 10.3%, treated verses untreated) and B2B2 subjects (6.5% verses 11.3% treated verses untreated respectively). Overall, for the combined B2 containing genotypes (B1B2 and B2B2 ) a reduction from 10.5% to 6.2% in critical events for the use of statin was seen (p=0.008). These observations clearly indicate a benefit with respect to cardiovascular endpoints of statin therapy for individuals having the B2 allele. However, a similar benefit was not seen for subjects with the B1B1 genotype. As seen for all combined endpoints, no difference between B2 homozygotes and heterozygotes was observed, again suggesting a dominant effect of haplotypes bearing the B2 allele.

The combined CV endpoints were further subdivided into CV death and non-fatal MI and these individual endpoints were analyzed separately by genotype and therapy. The result was that for cardiovascular death, a trend toward an event reduction was seen for patients treated with statin verses untreated patients with the B1B1 genotype (2.2% and 3.7% respectively). Although these data does not achieve statistical significance (p=0.27), it may nevertheless indicate some tendency of statin treatments to increase the risk of critical events for patients with the B1B1 genotype. Conversely, a significant statin treatment benefit was seen for B2 possessing individuals (5.2% verses 2.0%, untreated and treated respectively; p=0.006).

The occurrence of non-fatal MI subsequent to entrance into the study was also examined. Overall, the event rate was identical for treated and untreated patients (5.6% and 5.6%). When reanalyzed by Taq 1B genotype, there was a non-significant (p=0.1) trend towards more frequent non-fatal MI among B1 homozygotes on statin therapy (7.7%) as compared with untreated patients (4.8%). Conversely, a nonsignificant trend towards a reduced event rate was seen among B1B2 individuals receiving treatment (4.5%) compared with untreated (5.6%); a similar trend was also seen for B2B2 individuals (4.3% verses 6.8%, treated and untreated, respectfully). Overall, a 24% reduction in the rate of non-fatal MI was seen for B2 possessing patients, but this did not achieve statistical significance (p=0.24). Although there is a lack of statistical significance, the trended reduction of non-fatal MI for B2 heterozygotes and homozygotes continues to suggest that the therapeutic benefit of statin treatment is exclusive to B2 individuals.

The work of the inventors has shown that a subset of patients (B1B1 individuals) do not benefit from the statin medication. Moreover, the study suggests that for these individuals statin treatment may actually increase their risk of critical events. In sum, the inventors have found that a significant predictor of response to statin therapy is the CETP Taq 1 polymorphism genotype. Moreover, the inventors believes that similar relationships between specific genetic characteristics and pharmacological treatments exist and that the consideration of the connections between specific genetic characteristics and specific medical treatments will increase the quality of patient care while simultaneously reducing the cost of such care.

FIG. 1 shows the preferred steps of the application of the inventor's study to the treatment of patients, of this invention. Initially, a patient is presented 101 for clinical evaluation. A first test 102 is made to determine whether the patient is diagnosed with either a cardiovascular risk factor or cardiovascular disease. if the patient is not diagnosed with either a cardiovascular risk factor or cardiovascular disease, the process ends 108. If the patient is diagnosed with either a cardiovascular risk factor or cardiovascular disease, then a second test 103 is made to determine whether treatment with medication is indicated according to ordinary medical practice. If treatment with medication is not ordinarily indicated than the process ends 108. If the result of the second test 103 is that medication treatment would ordinarily be prescribed, then the patient's DNA/RNA is tested 104 to identify relevant genetic genotypes. A third test 105 is made to determine whether there is an identified genetic sensitivity benefit to the ordinary medication treatment. If no identified genetic sensitivity benefit of the medical treatment is determined then the medication is either not prescribed or is given in a lower dosage 107 as appropriate. For example, if it is found that the patient with the identified genetic characteristic would have a likely adverse reaction to the medication, then the medication is not given. Alternatively, if it is found that the patient with the identified genetic characteristic may not benefit but would not likely be harmed by the medication, a lower dosage may be appropriate. If an identified genetic sensitivity medication benefit is determined then the prescription is given, possibly in a higher dosage, 106 as medically appropriate. For example, if the DNA/RNA test 104 is correlated to an improved response to the proposed medication, the medication may be given in a higher than standard dosage. After the determinations of medication treatment and dosage 106, 107 are made, the process ends 108.

FIG. 2 is a table showing the baseline characteristics of patients categorized by treatment status and by Taq 1B genotype in the inventors' study.

FIG. 3 is a chart showing the frequency of events (all-cause mortality and non-fatal MI) for statin-treated and untreated patients further separated into the three possible CETP Taq 1B genotypes. This chart shows that the benefits of statin treatment for patients having the B1B1 genotype is statistically insignificant, as compared to that of patients having either the B1B2 or the B2B2 genotypes.

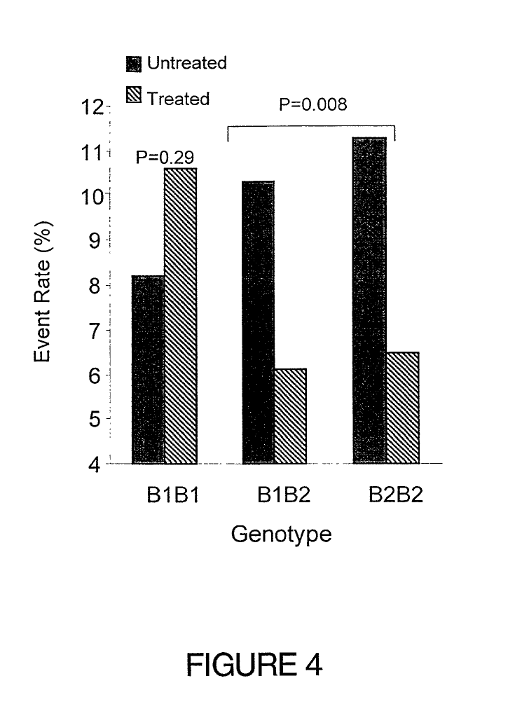

FIG. 4 is a chart showing the frequency of cardiovascular events (cardiovascular-related death and non-fatal MI) for statin-treated and untreated patients further separated into three possible CETP Taq 1B genotypes. This chart shows that, unlike patients with the B1B2 and the B2B2 genotypes, those with the B1B1 genotype, who are treated with statin, have a tendency, however potentially statistically insignificant, toward an increased rate of occurrence of cardiovascular-related death and non-fatal MI.

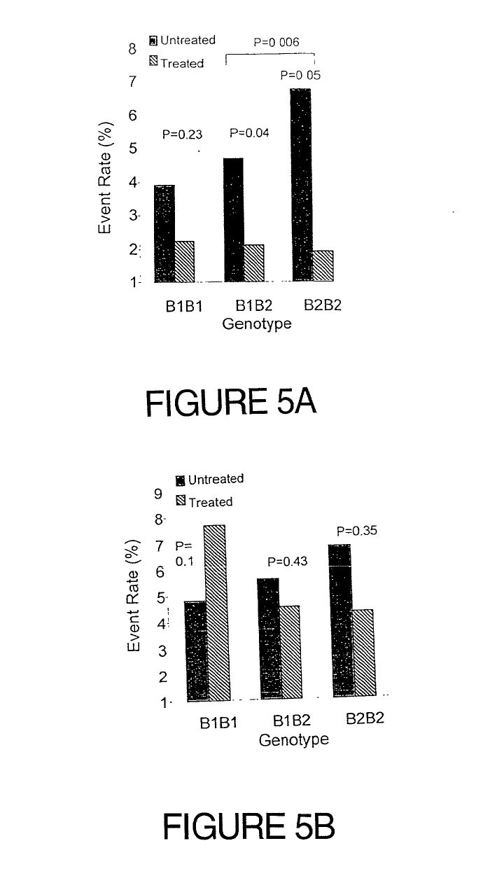

FIGS. 5A and 5B are charts showing subgroup analysis of the frequency of cardiovascular death (FIG. 5A) and non-fatal MI (FIG. 5B) for statin-treated and untreated patients further separated into the three possible CETP Taq 1B genotypes.

FIG. 5A shows that patients having each of the genotypes, B1B1; B1B2; B2B2; having statin treatment have a reduced event rate for cardiovascular death. Although, the data for B1B1 is not statistically significant. FIG. 5B shows that patients with the B1B2 and B2B2 genotypes who have statin treatments, have a reduced rate of non-fatal MI occurrences, while patients with the B1B1 genotype have an increased, although statistically insignificant, likelihood of non-fatal MI events.

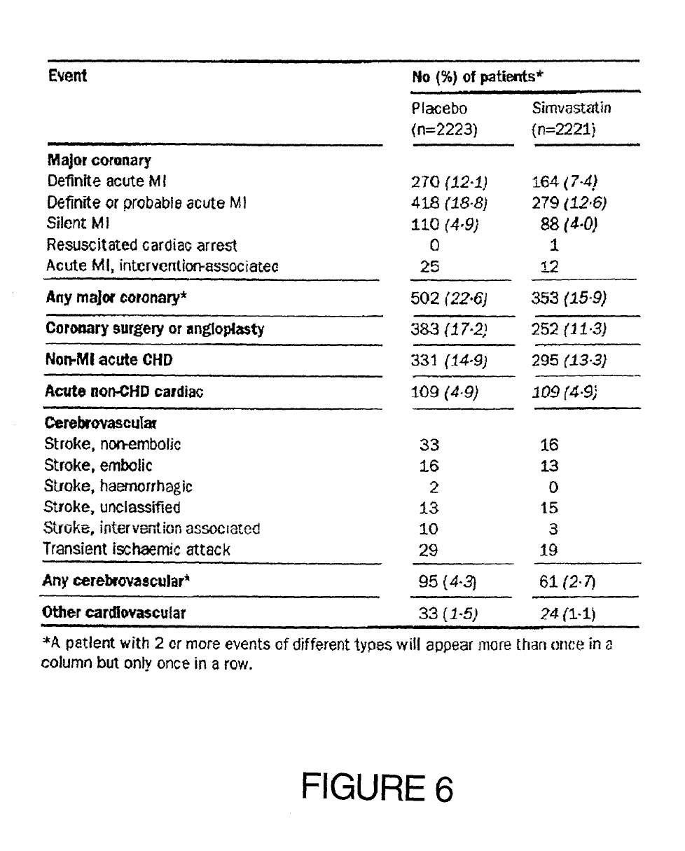

FIG. 6 is a table showing patients with nonfatal cardiovascular events during follow-up. The type of critical event is shown in the first column 601, while the number of patients having placebo treatment suffering from each event is shown in second column 602. The number of patients having simvastatin treatment who suffer from each event is shown in the third column 603. In general, this table shows that statin treatment tends to reduce the occurrence critical events, although some events such as acute non-CHD cardiac arrest do not appear to be reduced by statin treatment.

The described embodiments of this invention are to be considered in all respects only as illustrative and not as restrictive. Although the embodiments described herein include specific steps and describe a specific present application/example of the invention, the invention is not limited thereto. The scope of the invention is, therefore, indicated by the appended claims rather than by the foregoing description. All changes, which come within the meaning and range of equivalency of the claims, are to be embraced within their scope.

1. A process for the evaluation and prescription of pharmacological treatments to medical conditions, comprising:
(A) receiving a patient for medical treatment;
(B)determining if said received patient has a condition related to a disease;
(C)determining if treatment with a medicine is appropriate;
(D)testing the DNA of said patient to determine whether said patient has a particular DNA characteristic;
(E)evaluating said DNA characteristic of said patient to determine if said medicine has an effectiveness correlated to said DNA characteristic; and
(F)prescribing an appropriate quantity of said medicine.

2. A process for the evaluation and prescription of pharmacological treatments to medical conditions, as recited in claim 1, wherein said condition related to a disease is a risk factor.

3. A process for the evaluation and prescription of pharmacological treatments to medical conditions, as recited in claim 1, wherein said condition related to a disease is a disease.

4. A process for the evaluation and prescription of pharmacological treatments to medical conditions, as recited in claim 2, wherein said risk factor is a risk factor for cardiovascular disease.

5. A process for the evaluation and prescription of pharmacological treatments to medical conditions, as recited in claim 3, wherein said disease is cardiovascular disease.

6. A process for the evaluation and prescription of pharmacological treatments to medical conditions, as recited in claim 1, wherein said DNA test identifies whether said patient has the B1B1 genotype

7. A process for the evaluation and prescription of pharmacological treatments to medical conditions, as recited in claim 1, wherein said medication is a statin.
